SEQUENCE LISTING (4 sequences)

SEQ ID NO: 1 - protein, 13 aa, synthetic construct
  SITE 1: note = N-terminus acetylation
  VARIANT 2: note = X is homocysteine ("Hcy") or cystathionine ("Cth") that forms a cyclic thioether bond with a non-consecutive amino acid
  DISULFID 1..6: note = Intrachain disulfide bond
  CROSSLNK 2..10: note = Thioether bond
  DISULFID 5..13: note = Intrachain disulfide bond
CXELCCNVACYGC

SEQ ID NO: 2 - protein, 11 aa, synthetic construct
  SITE 8: note = Residue at position 8 is linked to dipeptide comprising an N-terminus acetylation, and C-terminus allyl ester, and creates a cystathionine thioether side chain bridge
  SITE 1: note = N-terminus fluorenylmethyloxycarbonyl (Fmoc)
ELCCNVACYGC

SEQ ID NO: 3 - protein, 11 aa, synthetic construct
  SITE 2..10: note = Thioether bond
  VARIANT 2: note = X is homocysteine ("Hcy") or cystathionine ("Cth") that forms a cyclic thioether bond with a non-consecutive amino acid
ELCCNVACYGC

SEQ ID NO: 4 - protein, 13 aa, synthetic construct
CXELCCNVACYGC

(19) United States
(12) Patent Application Publication
(10) Pub. No.: US 2025/0197449 A1
(43) Pub. Date: Jun. 19, 2025

(54) Synthetic Process for Production of Modified GCC Receptor Agonists

(71) Applicant: Ironwood Pharmaceuticals, Inc., Boston, MA (US)

(72) Inventors: Christopher LEITHEISER, Arlington, MA (US); Gunnar STAERKAER, Boston, MA (US); Ulf ANNBY, Boston, MA (US)

(21) Appl. No.: 18/711,044

(22) PCT Filed: Nov. 22, 2022
(86) PCT No.: PCT/US2022/080303

§ 371 Date: May 16, 2024

Related U.S. Application Data
(60) Provisional application No. 63/282,851, filed on Nov. 24, 2021.

(51) Int. Cl. C07K 7/64; C07K 1/00; C07K 1/06

(52) U.S. Cl. CPC C07K 7/64; C07K 1/006; C07K 1/061

(57) ABSTRACT

The present invention relates to methods of producing a synthetic peptide or pharmaceutically acceptable salts thereof of SEQ ID NO: 1.

### CROSS REFERENCE TO RELATED APPLICATIONS

The present application claims priority to and the benefit of U.S. Provisional Application No. 63/282,851, filed Nov. 24, 2021, the contents of which are herein incorporated by reference in their entirety.

### FIELD OF THE INVENTION

The present invention relates to methods of producing a synthetic peptide or pharmaceutically acceptable salts thereof of SEQ ID NO: 1.

### SEQUENCE LISTING

This application incorporates by reference in its entirety the Sequence Listing XML entitled “223355-519433.xml” (8.11 kilobytes) which was created Nov. 18, 2022 at 3:45 PM, and filed electronically herewith.

### BACKGROUND OF THE INVENTION

Interstitial cystitis/bladder pain syndrome (IC/BPS) is a chronic condition involving bladder pain usually accompanied by urinary urgency, increased frequency, and/or nocturia. IC/BPS is often misdiagnosed as a urinary tract infection and antibiotics are generally ineffective. It is estimated that 3-7% of women and 3-4% of men meet the definition of IC/BPS. There may be several contributing factors for the cause of IC/BPS, and it is unknown if IC/BPS is a primary disorder or the secondary result of another disorder [Hanno et al. 2015, 193; 1545-1553]. There are no diagnostic tests for IC/BPS and diagnosis is generally based on urinary symptoms of urgency and frequency accompanied by pain related to the bladder. Diagnosis is generally reserved until other diseases that could cause these symptoms are ruled out.

There are few approved therapies available for IC/BPS. Patients often begin treatment with non-pharmacological treatments (general relaxation, stress management, behavior modification, and physical therapy techniques). Due to the marginally effective therapies available for IC/BPS, many patients utilize off-label therapies including intravesical instillations (i.e. mixtures of medications delivered directly to the bladder through a catheter) to relieve their symptoms. A need exists for more effective, well tolerated treatments for IC/BPS.

A 13-amino-acid, guanylate cyclase C (GC-C) agonist synthetic peptide is being developed for the treatment of bladder pain associated with IC/BPS and, potentially, other visceral pain conditions in the abdominal region. To further the development of this peptide, a need exists for an efficient synthesis and purification process.

### SUMMARY OF THE INVENTION

The present invention relates to a method of producing a synthetic peptide, or a pharmaceutically acceptable salt thereof. The method having the steps of (i) chemically synthesizing a linear peptide having its C-terminal bound to a solid phase support and a protected amine group at its N-terminal using a plurality of amino acids and at least one polyamino acid synthon, the linear peptide having protecting groups in one or more amino acids and/or the at least one polyamino acid synthon; wherein the synthon has at least one amine group acetylated and at least one carboxylic acid protecting group; (ii) removing the carboxylic acid protecting group of the synthon and the protecting group from the amine group at the N-terminal of the linear peptide to form a partially unprotected solid phase support-bound peptide having an unprotected amine group and an unprotected carboxylic acid group; (iii) coupling the unprotected amine group and the unprotected carboxylic acid group to form a cyclized solid phase support-bound peptide; (iv) cleaving the cyclized solid phase support-bound peptide from the solid phase support to generate a cyclized protected peptide; (v) globally deprotecting the cyclized protected peptide to obtain a globally deprotected peptide; (vi) folding the globally deprotected peptide to form one or more additional crosslinks to obtain the synthetic peptide; and (vii) purifying the synthetic peptide. The synthetic peptide comprises the amino acid sequence: Ac-Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1). The synthetic peptide contains a covalent bond between the following amino acid residues: Cys₁ and Cys₆, Cth₂ and Cys₁₀, and Cys₅ and Cys₁₃.

### BRIEF DESCRIPTION OF THE FIGURES

FIG. 1 shows an exemplary flow diagram for the manufacture of the linear synthetic peptide of step (i) of the method described herein.

### DETAILED DESCRIPTION OF THE INVENTION

A method of producing a synthetic peptide, or a pharmaceutically acceptable salt thereof is described herein. The method described herein comprises:
(i) chemically synthesizing a linear peptide having its C-terminal bound to a solid phase support and a protected amine group at its N-terminal using a plurality of amino acids and at least one polyamino acid synthon, the linear peptide having protecting groups in one or more amino acids and/or the at least one polyamino acid synthon;
wherein the synthon has at least one amine group acetylated and at least one carboxylic acid protecting group;
(ii) removing the carboxylic acid protecting group of the synthon and the protecting group from the amine group at the N-terminal of the linear peptide to form a partially unprotected solid phase support-bound peptide having an unprotected amine group and an unprotected carboxylic acid group;
(iii) coupling the unprotected amine group and the unprotected carboxylic acid group to form a cyclized solid phase support-bound peptide;
(iv) cleaving the cyclized solid phase support-bound peptide from the solid phase support to generate a cyclized protected peptide;
(v) globally deprotecting the cyclized protected peptide to obtain a globally deprotected peptide;
(vi) folding the globally deprotected peptide to form one or more additional crosslinks to obtain the synthetic peptide; and
(vii) purifying the synthetic peptide;
wherein the synthetic peptide comprises the amino acid sequence: Ac-Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1);
wherein the synthetic peptide contains a covalent bond between the following amino acid residues: a) Cys₁ and Cys₆, b) Cth₂ and Cys₁₀, and c) Cys₅ and Cys₁₃.

### Definitions

As used herein, “Cth” represents cystathionine which has two α-amino carboxyl groups, designated “1” and “2” in Scheme 1, which can form peptide bonds.

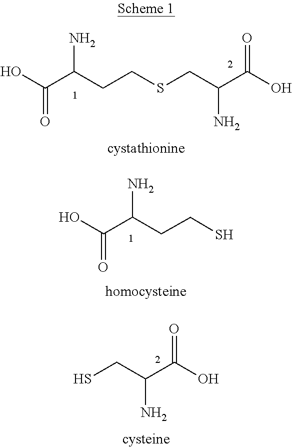 [SMILES: NC(CCSCC(N)C(=O)O)C(=O)O ; NC(CCS)C(=O)O ; NC(CS)C(=O)O]

However, to facilitate the use of the three letter amino acid code in describing a peptide sequence, when a cyclic peptide sequence is created by forming a peptide bond with each of the α-amino carboxyl group (designated “1” and “2”) at non-consecutive positions in the peptide sequence, thereby creating a cyclic thioether bridge, the peptide bond formed by the α-amino carboxyl group at position 1 is designated “Cth,” whereas the peptide bond formed by the α-amino carboxyl group at position 2 is designated “Cys.” See the section entitled “Synthetic Peptide” for further details.

As used herein, “Hcy” or “Hcys” represents homocysteine as shown in Scheme 1. As can be seen from Scheme 1, cystathionine can be viewed as a combination of homocysteine and cysteine where their side chains share a sulfur atom. Therefore, an alternative method of designating a cyclic peptide sequence created by forming a peptide bond with each of the α-amino carboxyl group of cystathionine at non-consecutive positions in the peptide sequence is by designating the peptide linkage formed by the α-amino carboxyl group at position 1 “Hcy” and the peptide linkage formed by the α-amino carboxyl group at position 2 “Cys”.

As used herein, unless other indicated, “pharmaceutically acceptable” means biologically or pharmacologically compatible for in vivo use in animals or humans, and preferably means, approved by a regulatory agency of the Federal or a state government or listed in the U.S. Pharmacopeia or other generally recognized pharmacopeia for use in animals, and more particularly in humans.

As used here, unless otherwise indicated, the terms “about” and “approximately” mean within an acceptable error range for the particular value as determined by one of ordinary skill in the art, which will depend, in part, on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%. Alternatively, particularly with respect to biological systems or processes, the term can mean within an order of magnitude, preferably within 5-fold, and more preferably within 2-fold, of a value. Particular values are described in the application and claims, unless otherwise stated the term “about” means within an acceptable error range for the particular value.

### Synthetic Peptide

In some embodiments, the synthetic peptide produced by the methods of the present disclosure can be linearly represented as Ac-Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein “Ac” indicates that the N-terminus amine group is acetylated.

The synthetic peptide of SEQ ID NO: 1 contains four cysteine residues that form two disulfide bonds, and a cystathionine (Cth) unit (combining homocysteine and cysteine, which share the sidechain sulfur atom) providing an internal sulfide (or thioether) bond, with the defined connectivity (Cys₁-Cys₆, Cys₅-Cys₁₃, Cth₂-Cys₁₀).

For purposes of the present description, the two parts of the linear sequence are designated as Cth₂ and Cys₁₀, where the thioether bond connects the sulfur a homocysteine (Hcy) side chain and a carbon of a des-SH cysteine side chain: this double amino-acid corresponds to a cystathionine (Cth) residue, but the proposed designation facilitates the description when using the 3-letter code designation of the residues where the peptide linkage formed by the α-amino carboxyl group of position 1 of the cystathionine is designated “Cth” and the peptide linkage formed by α-amino carboxyl group of position 2 is designated “Cys.”

Alternatively, and for purposes of the present description, the two parts of the building blocks may be designated, respectively, as [Hcy] and [Cys] where the sulfur of the homocysteine (Hcy) side chain is shared with a side chain of a cysteine (Cys) to form a thioether bridge: this double amino-acid corresponds to a cystathionine (Cth) residue but the proposed designation facilitates the description when using the 3-letter code designation of the residues. Using this alternative nomenclature SEQ ID NO 1 is represented as follows: Ac-Cys₁ Hcy₂ Glu₃ Leu₄ Cys₅ Cys₆ Asn₇ Val₈ Ala₉ Cys₁₀ Tyr₁₁ Gly₁₂ Cys₁₃ (SEQ ID NO: 1).

In some embodiments, the designation of Cth₂-Cys₁₀, or any variation thereof, is meant to describe the linkage between the side chains of two non-consecutive amino acids in SEQ ID NO 1 which forms a thioether bridge as shown below:

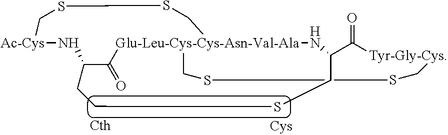

(SEQ ID NO: 1) In some embodiments, the designation of Cth₂-Cys₁₀, or any variation thereof, describes cystathionine which forms a peptide bond at positions 2 and 10 of the synthetic peptide and forms a thioether bridge.

In some embodiments, the synthetic peptide of SEQ ID NO: 1 can be represented by the formula:

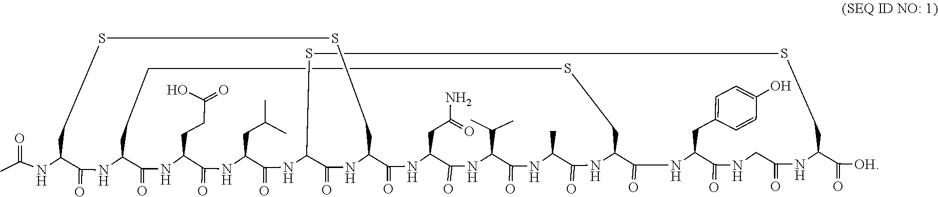

### Method of Producing a Synthetic Peptide

The method described herein begins by (i) chemically synthesizing a linear peptide having its C-terminal bound to a solid phase support and a protected amine group at its N-terminal using a plurality of amino acids and at least one polyamino acid synthon, the linear peptide having protecting groups in one or more amino acids and/or the at least one polyamino acid synthon. In some embodiments, the synthon has at least one amine group acetylated and at least one carboxylic acid protecting group.

In some embodiments, the solid phase support is selected from the group consisting of Wang resins, Trityl resins, and Rink resins.

In some embodiments, the solid phase support has a loading of about 0.10 mmol/g, about 0.20 mmol/g, about 0.30 mmol/g, about 0.40 mmol/g, about 0.50 mmol/g, about 0.60 mmol/g, about 0.70 mmol/g, about 0.80 mmol/g, about 0.90 mmol/g, or about 1.00 mmol/g. In some embodiments, the solid phase has a loading of about 0.70 mmol/g. In some embodiments, the solid phase has a loading of about 0.90 mmol/g.

In some embodiments, the polyamino acid synthon is a compound represented by the formula:

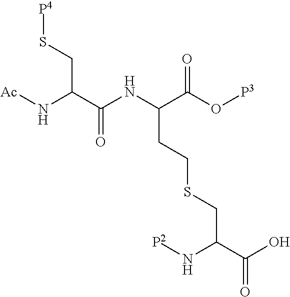

where P² is an amine protecting group; P³ is a carboxylic acid protecting group; and P⁴ is a thiol protecting group.

In some embodiments, the protecting groups are selected from the group consisting of fluorenylmethyloxycarbonyl (Fmoc), tert-butyloxycarbonyl (Boc), carboxybenzyl (Cbz), trityl, methyl, ethyl, tert-Butyl, allyl, 2,4-dimethoxybenzyl (Dmb), 9-fluorenylmethyl (Fm), benzyl (Bn), tert-butyldimethylsilyl, allyloxycarbonyl (alloc), tert-butyloxycarbonyl, acetamidomethyl (Acm), 3-nitro-2-pyridine sulfenyl (NPYS) and 2-pyridine-sulfenyl (Pyr).

In some embodiments, the amine protecting groups, P², is selected from the group consisting of fluorenylmethyloxycarbonyl (Fmoc), tert-butyloxycarbonyl (Boc), and carboxybenzyl (Cbz). In some embodiments, P² is a 9-fluorenylmethoxycarbonyl (Fmoc) protecting group.

In some embodiments, the carboxylic acid protecting group, P³, is selected from the group consisting of methyl, ethyl, tert-Butyl, allyl, 2,4-dimethoxybenzyl (Dmb), 9-fluorenylmethyl (Fm), benzyl (Bn). In some embodiments, P³ is an allyl protecting group.

In some embodiments, P⁴ is a trityl protecting group.

In some embodiments, the subunits of the polyamino acid synthon have a D-configuration, e.g., the synthon is a D-Enantiomer. In some embodiments, the polyamino acid synthon with subunits of a D-configuration can be represented by the following formula:

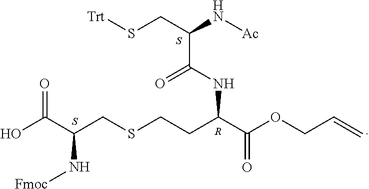

In some embodiments, the subunits of the polyamino acid synthon have an L-configuration, e.g., the synthon is an L-Enantiomer. In some embodiments, the polyamino acid synthon with subunits of a L-configuration can be represented by the following formula:

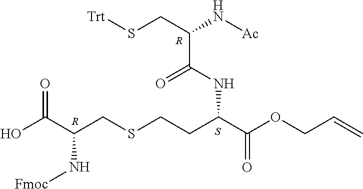

In some embodiments, the subunits of the polyamino acid synthon have both a D-configuration and an L-configuration.

In some embodiments, the amino acid side chains of the linear peptide have a protecting group. In some embodiments, the amino acid side chain protecting groups are selected from the group consisting of tert-Butyl (tBu), trityl (Trt), allyl, cyclohexyl, 2-phenylisopropyl, acetamidomethyl (Acm), benzyl (Bzl), 4-methylbenzyl (4-MeBzl), 4-methoxybenzyl (4-MeOBzl), 9-fluorenylmethyl (Fm), tert-butylthio (t-Buthio), 4-methoxytrityl (Mint), xanthyl (Xan), 2,6-Dichlorobenzyl (2,6-Cl₂Bzl), and 2-bromobenzylcarbonate (2-BrZ). In some embodiments, the amino acid side chain protecting group is tert-Butyl (tBu) or trityl (Trt).

In some embodiments, the amino acid side chains of the linear peptide that have a protecting group on their side chains are Cys₁, Glu₃, Cys₅, Cys₆, Asn₇, Tyr₁₁, and Cys₁₃ of SEQ ID NO: 1.

In some embodiments, the plurality of amino acids and the synthon are coupled by a carbodiimide-mediated reaction or by a reaction mediated by a non-carbodiimide coupling agents: 1-[Bis(dimethylamino)methylene]-1H-1,2,3-triazolo[4,5-b]pyridinium 3-oxide hexafluorophosphate (HATU), (2-(1H-benzotriazol-1-yl)-1,1,3,3-tetramethyluronium hexafluorophosphate (HBTU), 1H-Benzotriazolium 1-[bis(dimethyl-amino)methylene]-5-chloro-hexafluorophosphate (1-),3-oxide (HCTU), O-(Benzotriazol-1-yl)-N,N,N′,N′-tetramethyluronium tetrafluoroborate (TBTU), benzotriazol-1-yloxytripyrrolidinophosphonium hexafluorophosphate (PyBOP), 7-Azabenzotriazol-1-yloxy)tripyrrolidinophosphonium hexafluorophosphate (PyAOP), or propanephosphonic acid anhydride (T3P) to form the linear peptide of step (i).

In some embodiments, at least one amino acid from the plurality of peptides and/or the synthon are coupled by a carbodiimide-mediated reaction to form the linear peptide of step (i). In some embodiments, the carbodiimide is selected from the group consisting of diisopropylcarbodiimide (DIC), dicyclohexylcarbodiimide (DCC), and 1-Ethyl-3-(3-dimethylaminopropyl)carbodiimide (EDC). In some embodiments, the carbodiimide is DIC.

In some embodiments, the carbodiimide-mediated reaction further comprises an antioxidant. In some embodiments, the antioxidant is a soluble thiourea or thiol compound. In some embodiments, the antioxidant is 1,3-diisopropyl-2 thiourea (DITU), or dithiothreitol.

In some embodiments, at least one amino acid is coupled by a non-carbodiimide coupling agent. In some embodiments, the cyclization coupling reaction is mediated by a non-carbodiimide coupling agent. In some embodiments, the non-carbodiimide coupling agent used in the cyclization coupling reaction is HATU.

The linear peptide of step (i) may be referred to in this application as a “linear 13-mer” and can be represented by the following formula:

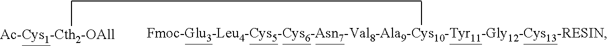

(SEQ ID NO: 2) wherein the cystathionine thioether side chain bridge (—CH₂—CH₂—S—CH₂—) is represented by “ [symbol] ” In some embodiments, one or more of the side chains of the underlined amino acids are protected. In some embodiments, the side chains of all the underlined amino acids are protected.

After the linear peptide is formed, the (ii) carboxylic acid protecting group of the synthon and the protecting group from the amine group at the N-terminal of the linear peptide are removed to form a partially unprotected solid phase support-bound peptide having an unprotected amine group and an unprotected carboxylic acid group. In some embodiments, the deprotection is achieved with Pd(PPh₃)₄ and 1,3-DMBA in DMF.

In some embodiments, the partially unprotected solid phase support-bound peptide can be represented by the following formula:

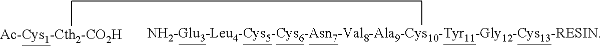

(SEQ ID NO: 3) Following the partial deprotection, (iii) the unprotected amine group and the unprotected carboxylic acid group are coupled to form a cyclized solid phase support-bound peptide.

In some embodiments, the cyclized solid phase support-bound peptide can be represented by the following formula:

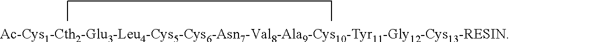

(SEQ ID NO: 4) The cyclized solid phase support-bound peptide is (iv) cleaved from the solid phase support to generate a cyclized protected peptide.

In some embodiments, the linear peptide is cleaved from the resin via a dilute acidic treatment. In some embodiments, the dilute acidic treatment preserves the side chain protecting groups and the protecting groups of the polyamino acid synthon. In some embodiments, the dilute acidic treatment is a weak acid solution, for example, trifluoroacetic acid (TFA). In some embodiments, the dilute acid solution is a trifluoroacetic acid (TFA) solution. In some embodiments, the dilute acid solution is a 1% trifluoroacetic acid (TFA) in dichloromethane (DCM) solution.

Once the cyclized protected peptide is cleaved from the resin, the peptide is (v) globally deprotected to obtain a globally deprotected peptide. In some embodiments, the global deprotection step (v) comprises addition of a cocktail comprising at least ammonium iodide (NH₄I). In some embodiments, the global deprotection step (v) comprises addition of a cocktail comprising at least ammonium iodide (NH₄I) and triisopropylsilane.

Once the cyclized peptide is globally deprotected, the peptide is (vi) folded to form one or more additional crosslinks to obtain the synthetic peptide of Ac-Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1).

As used herein, “folding” and “oxidation” may refer to the same step, as the folding is achieved via oxidation of the cysteine residues of SEQ ID NO: 1. In some embodiments, the folding step (vi) is achieved via an iodine- or alkaline-mediated oxidation. In some embodiments, the alkaline-mediated oxidation is a dimethylsulfoxide (DMSO)- or an N-Methyl-2-pyrrolidone (NMP)-mediated oxidation.

In some embodiments, the synthetic peptide contains a covalent bond between the following amino acid residues of the synthetic peptide: Cys₁ and Cys₆, Cth₂ and Cys₁₀, and Cys₅ and Cys₁₃. In some embodiments, the covalent bond between Cys₁ and Cys₆ and Cys₅ and Cys₁₃ is a disulfide bond. In some embodiments, the covalent bond between Cth₂ and Cys₁₀ is a thioether bond.

Following the folding of the globally deprotected peptide, the synthetic peptide of SEQ ID NO: 1 is purified.

In some embodiments, the synthetic peptide of SEQ ID NO: 1 can be represented by the following formula:

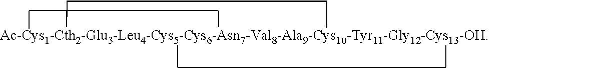

Also described herein is a method of preparing a synthetic peptide of Formula I:

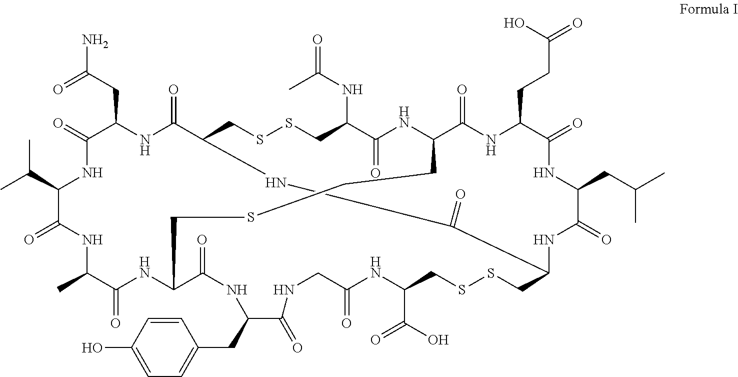 [SMILES: CC(=O)N[C@@H]1CSSC[C@@H]2NC(=O)[C@@H]3CSSC[C@@H](C(=O)O)NC(=O)CNC(=O)[C@@H](Cc4ccc(O)cc4)NC(=O)[C@@H](CSCC[C@@H](NC1=O)C(=O)N[C@@H](CCC(=O)O)C(=O)N[C@@H](CC(C)C)C(=O)N3)NC(=O)[C@@H](C)NC(=O)[C@@H](C(C)C)NC(=O)[C@@H](CC(N)=O)NC2=O]

the method comprising:
   (i) coupling a C-terminal resin bound Tyr-Gly-Cys peptide having protected amino acid side chains to a polyamino acid synthon of Formula II:

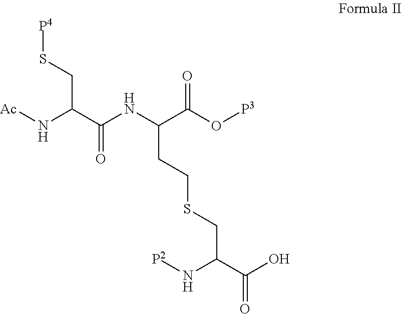

wherein:
   P² is an amine protecting group;
   P³ is a carboxylic acid protecting group; and
   P⁴ is a thiol protecting group;
to form a resin bound peptide of Formula III:

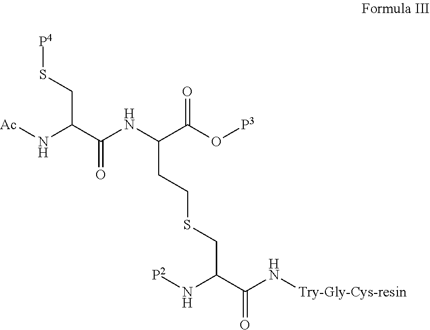

(ii) removing the P² protecting group of Formula III to obtain a resin bound peptide of Formula IV having an unprotected amine group:

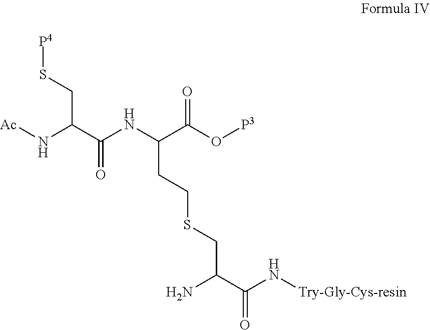

(iii) coupling a P²-alanine to the resin bound peptide of Formula IV via the free amine group of Formula IV to form a resin bound peptide of Formula V:

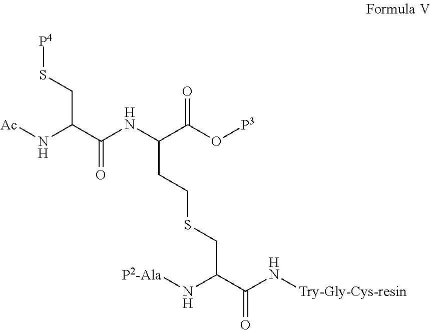

(iv) removing the P² protecting group of Formula V to obtain a free amine group, followed by coupling the free amine group to P²-amino acid, wherein the side chain of the amino acid may be protected;
(v) repeating step (iv) for five more times to form a resin bound peptide of Formula VI:

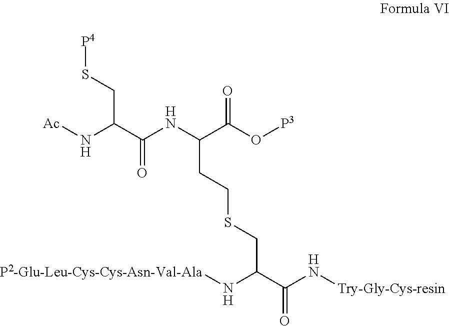

wherein at least one amino acid side chain is protected;
(vi) removing the P² protecting group and the P³ protecting group to obtain a free amine group and a free carboxylic acid group;
(vii) coupling the free amine group and the free carboxylic acid group, to obtain a cyclized peptide of Formula VII:

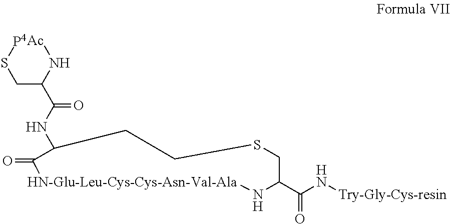

(viii) cleaving the peptide of Formula VII from the resin to obtain a cyclized peptide;
(ix) globally deprotecting the cyclized peptide to obtain a globally deprotected peptide; and
(x) folding the globally deprotected peptide by forming two disulfide bonds to obtain the synthetic peptide of Formula I.

In some embodiments, the Glu, Cys, Cys, Asn, Gly, and Cys residues of Formula VI have side chain protecting groups. In some embodiments, the amino acid side chain protecting groups are selected from the group consisting of tert-Butyl (tBu), trityl (Trt), allyl (All), cyclohexyl, 2-phenylisopropyl, acetamidomethyl (Acm), benzyl (Bzl), 4-methylbenzyl (4-MeBzl), 4-methoxybenzyl (4-MeOBzl), 9-fluorenylmethyl (Fm), tert-butylthio (t-Buthio), 4-methoxytrityl (Mint), xanthyl (Xan), 2,6-Dichlorobenzyl (2,6-Cl₂Bzl), and 2-bromobenzylcarbonate (2-BrZ). In some embodiments, the amino acid side chain protecting group is tert-Butyl (tBu) or trityl (Trt).

In some embodiments, P² is a protecting group selected from the group consisting of fluorenylrnethoxycarbonyl (Fmoc), tert-butyloxycarbonyl (Boc), carboxybenzyl (Cbz), and allyloxycarbonyl (Alloc). In some embodiments, P² is a fluorenvlmethoxycarbonyl (Fmoc) protecting group.

In some embodiments, P³ is a protecting group selected from the group consisting of methyl, ethyl, tert-Butyl, allyl (All), trityl, 2,4-dimethoxybenzyl (Dmb), 9-fluorenylmethyl (Fm), and benzyl (Bn). In some embodiments, P³ is an allyl (All) protecting group.

In some embodiments, P⁴ is a protecting group selected from the group consisting of acetamidomethyl (Acm), tert-butyl (t-But), 3-nitro-2-pyridine sulfenyl (NPYS), 2-pyridine-sulfenyl (Pyr), and trityl (Trt). In some embodiments, P⁴ is a trityl protecting group.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Abbreviations

AA: Amino acid
AAA: Amino acid analysis
Ac: Acetyl
AcOH: Acetic acid
All or Allyl: 2-propenyl
API Active Pharmaceutical Ingredient
BB: (Fmoc-Cys¹⁰*[Ac-Cys(Trt)-Hcys(S*)-OAll]-OH) which has the following structure:

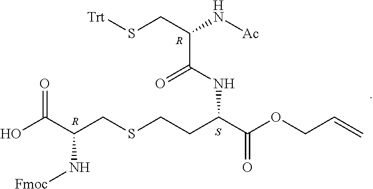

C18: Silica gel C18
DBU: 1,8-Diazabicyclo[5.4.0]undec-7-ene
DIC: Diisopropylcarbodiimide
DIPEA: Diisopropylethylamine
DITU: 1,3-Diisopropyl-2-thiourea
1,3-DMBA: 1,3-Dimethylbarbituric acid
DMF: N,N-Dimethylformamide
DMSO: Dimethylsulfoxide
DTT: Dithiothreitol
EtOH: Ethanol
eq: equivalent
Fmoc: 9-fluorenylmethoxycarbonyl
GC: Gas chromatography
GSH/GSSG: Glutathione (red./ox.)
HATU: 1H-1,2,3-Triazolo[4,5-b]pyridinium, 1-[bis(dimethylamino)methylene]-, 3-oxide, hexafluorophosphate(1-) (1:1)
HDPE: High density polyethylene
HPLC: High performance liquid chromatography
IPA: Isopropanol
LC: Liquid chromatography
MeCN: Acetonitrile
MeOH: Methanol
MTBE: Methyl tert-butyl ether
MS: Mass spectrometry
Mw: Molecular weight
NH₄OAc: Ammonium acetate
NMM: N-Methylmorpholine
NMP: N-methylpyrrolidone
NMR: Nuclear magnetic resonance
Oxyma: Ethyl(hydroxyimino)cyanoacetate
PPh₃: Triphenylphosphine
SEC: Size exclusion chromatography
SPPS: Solid phase peptide synthesis
tBu: tert-butyl
TFA: Trifluoroacetic acid
Thz: Thiazolidine
Thz(Me)₂: 2,2-Dimethylthiazolidine
TIS: Triisopropylsilane
Trt: Triphenylmethyl
Trt-H: Triphenylmethane
UV: Ultraviolet

### Example 1

### Manufacturing Process of Synthetic Peptide of SEQ ID NO: 1

The peptide of SEQ ID NO: 1, or a pharmaceutically acceptable salt thereof, was manufactured in compliance with Good Manufacturing Practice (GMP) regulations.

### Materials

Fmoc-D-Cys(Trt)-OH (>97.0%, HPLC) and TFA (>99.0%, HPLC) for HPLC analyses were purchased from Sigma-Aldrich. Fmoc-Thz-OH (99.6%, HPLC) and Fmoc-Thz(Me)₂-OH (>99.3%, HPLC; 99.7% ee) were obtained from PepTech. BB (>98.5%, HPLC) was custom synthesized. The identity of BB, L-enantiomer, was confirmed by NMR and chiral analysis. DITU from Molekula, DMSO (pa) from Riedel-de Hahn, and O-methylhydroxylamine hydrochloride from Merck were used. GSH and GSSG were obtained from Sigma-Aldrich. All other reagents, amino acid derivatives, 2-CTC resin, and solvents were taken from the warehouse. All materials were used as received. Purified water was used.

### Equipment

### Synthetic Equipment

For small scale SPPS and cleavages/deprotections special stoppered syringes with filters were used. Syringes with their contents were shaken automatically at ambient temperature (20-25° C.).

Larger scale SPPS utilized jacketed reactors with overhead stirrers. Temperature was controlled in jacketed reactors with a cryostat (Julabo).

Cleavages/deprotections starting from >5 g peptide resin were made in glassware or closed HDPE jars.

Iodine mediated oxidation (disulphide formation) was made with the aid of syringe pump and suitable TEFLON tubing.

Otherwise, standard lab equipment/utensils were used for synthetic work.

Occasionally, a Beckman centrifuge (3750-4000 rpm, 10 min) was used for small scale precipitations of cleaved/deprotected peptides.

### Analytical Equipment

HPLC was run using an Agilent system equipped with a XSelect Peptide 130 A 150×4.6 mm 2.5μ column at 30° C. Mob. phase A: 0.1% TFA(aq); mob. phase B: 0.1% TFA(MeCN)

LC/MS used the same column on a Thermo Scientific Vanquish Horizon UHPLC, connected to an ESI Thermo Q-Exactive MS Spectrometer. Chromeleon software was used for HPLC/MS evaluations.

Chiral AAA was made at C.A.T. GmbH & Co. Chromatographie and Analysentechnik KG, Tubingen, Germany.

NMR analyses were performed by RED GLEAD DISCOVERY AB, Medicon Village, 223 81 Lund, Sweden.

### Peptide Synthesis

### SPPS

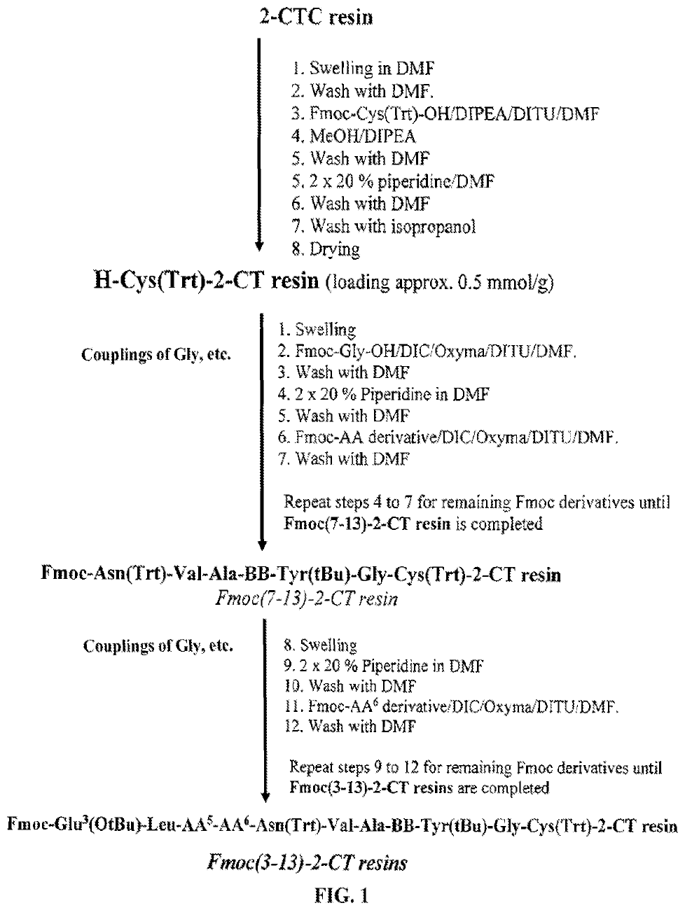

FIG. 1 shows a flow diagram of the SPPS synthesis of the solid-bound 13-mer.

Standard protocols were used. All peptide resins were stored in a freezer after drying.

### L- or D-H-Cys-2-CT Resins

L- or D-Cys was attached to 2-CTC resin in a SPPS reactor with a glass filter for about 5 h at 20-25° C., and remaining coupling sites on the resin were capped with methanol. After draining/washing, the Fmoc group was removed by treatment with 20% (v/v) piperidine in DMF (2×10 min). The resin was washed with DMF until negative chloranil test indicating the removal of piperidine, then with isopropanol, and finally dried in vacuo at 20-25° C. for 1-2 days.
L-H-Cys-2-CT resin; 102.0 g, 0.42 mmol/g (triphenylmethane)
D-H-Cys-2-CT resin; 14.5 g, 0.40 mmol/g (triphenylmethane)

### Fmoc(7-13)-2-CT Resins

For the L-Cys₁₃ version, a jacketed reactor was used. Reactions were made at 20° C. (T_{jacket}), 20-25° C. in the D-Cys₁₃ case.

Couplings were made either in DMF or NMP. Relative to the Cys₁₃ loading, about 2.0 eq. of AA derivatives used, except for BB (1.5 eq.). About 2.2 eq. of Oxyma/DIC, and about 0.2 eq. DITU were used. Glycine was preactivated for 1 h. Additional DIC (about 2.2 eq.) was generally added after some time. Total coupling times were generally 2-3 h, and around 5 h for BB.

The glycine was preactivated to speed up the Gly coupling, and thus avoid unintentional detachment of Cys from the acid sensitive 2-CT resin by slightly acidic Oxyma or amino acid, potentially leading to endo- and des-Cys.

After each coupling, the reactor was drained and the resin washed with DMF. When syntheses were paused overnight, peptide resins were stored (5-10° C.) in the reactors. Operating temperature was increased before continued synthesis.

The Fmoc group was removed by treatment with 20% (v/v) piperidine in DMF (2×10 min), and the resin thoroughly washed until negative chloranil test, which indicated complete removal of piperidine.

When the sequence was complete, washing with DMF and isopropanol was made. The resin was then dried in vacuo at 20-25° C. for 1-2 days.

One of Cys_{(5 or 6)} was incorporated as a pseudoproline, to facilitate the cyclization by the cis induced conformation. Suitable derivatives chosen can be Fmoc-L-Thz-OH (CAS no. [133054-21-4]) and Fmoc-L-Thz(Me₂)-OH (CAS no. [873842-06-9]). Thz could substitute Cys(Trt) in position 6 or 5, or in both positions. Accordingly, four different Fmoc(3-13)-2-CT resin peptides were synthesized as shown below:

| | |
|---|---|
| Fmoc-Glu(OtBu)-Leu-Cys(Trt)-Cys(Trt)-Asn(Trt)- | 8.82 g (98%); 0.158 |
| Val-Ala-BB-Tyr(tBu)-Gly-Cys(Trt)-2CT resin | mmol/g (95.8%) |
| Fmoc-Glu(OtBu)-Leu- [symbol] -Cys(Trt)-Asn(Trt)-Val- | 8.15 g (94%); 0.120 |
| Ala-BB-Tyr(tBu)-Gly-Cys(Trt)-2CT resin | mmol/g (69.6%) |
| Fmoc-Glu(OtBu)-Leu-Cys(Trt)- [symbol] -Asn(Trt)-Val- | 8.11 g (94%); 0.145 |
| Ala-BB-Tyr(tBu)-Gly-Cys(Trt)-2CT resin | mmol/g (83.5%) |
| Fmoc-Glu(OtBu)-Leu- [symbol] -Asn(Trt)-Val-Ala-BB- | 7.79 g (94%); 0.145 |
| Tyr(tBu)-Gly-Cys(Trt)-2CT resin | mmol/g (80.6%) |

[symbol]

indicates data missing or illegible when filed

Yield based on theoretical product weight.

### Ring-Closures Lactamisations

Fmoc(3-13)-2-CT resins were swelled with DMF in syringes. After draining, Fmoc was removed with 20% piperidine (>10 min+>20 min). The resins were thoroughly washed until negative chloranil test. The allyl ester of Hcys was converted into the acid overnight with about 10 mol % Pd(PPh₃)₄ and about 10 eq of 1,3-DMBA in DMF. The resins were drained and washed with DMF, and ring-closure was made with about 2 eq of HATU and 2.7 eq of NMM. Kaiser tests indicated complete reactions within 4-5 h. Final washing and drying was made as described above.

### Cleavages/Deprotections

Analytical scale cleavages/deprotections were made as described below in syringes. The TFA solutions were filtered into ice cold (−18° C.) diethyl ether. The precipitate was centrifuged, the supernatant decanted, the residues triturated with diethyl ether, and the solids again centrifuged.

### Preparative Scale (Ac-Cys(H)-Hcys(S1-Glu-Leu-Cys(H)-Cys(H)-Asn-Val-Ala-C Tyr-Gly-Cys(H)—OH).

Ring-closed peptide resin Cys_{5 and 6}(Trt) (5.41 g) was mixed with 1.8 g DTT, 1.7 g NH₄I, 4.5 mL TIS, 1.8 mL water, and 50 mL TFA. The mixture was agitated for 3 h. The resin was filtered off, washed with 2×15 mL TFA. The combined filtrates were cooled (10° C.), and ice cold (−18° C.) diethyl ether (360 mL) was added portionwise with stirring during 10-15 min (T. 23° C.). Stirring was continued for about 5 min at 0° C. The solid was filtered (16-40 μm) and washed with diethylether (2×100 mL). It was then dried in vacuo for 2 days at 20-25° C. This yielded 1.80 g of crude cyclic peptide (free —SH).

### Oxidations (Formation of S—S Bridges)

These experiments were made with the crude cyclic peptide (free —SH) obtained from Fmoc-Glu(OtBu)-Leu-Cys(Trt)-Cys(Trt)-Asn(Trt)-Val-Ala-BB-Tyr(tBu)-Gly-Cys(Trt)-2CTC-Resin. All oxidations were made at 20-25° C.

### Iodine Oxidation

A solution of the crude cyclized peptide in DMSO (10 g/L) was made. The solution was diluted 10 times with 20% MeCN (aq), and became slightly turbid (pH about 4). To this solution iodine in MeCN (1% w/v) was added dropwise over about 1 h, until a yellow/brown color persisted. Iodine was quenched with aqueous ascorbic acid (0.5 M). pH was raised to 7-7.5 with 3.5% NH₃ (aq). GSH/GSSG was added (1-3.5 mM) at different points in time.

### DMSO Oxidations

Clear solutions were made with DMSO. These were the diluted to 30% DMSO with water (pH about 4) to a concentration of 1-3.3 g/L. This slightly turbid mixture was either stirred at 60° C., or pH adjusted to 7.5-8 with 3.5% NH₃ (aq). At the latter pH, the solution became completely clear again. GSH/GSSG was optionally added.

### NMP Oxidation

A clear solution of 0.5 g crude/mL NMP was made.

### Results and Discussion

### H-Cys(Trt)-2-CT and Fmoc(7-13)-2-CT Resins

A Cys loading of 0.5 mmol/g resin was aimed at. Consumption of Fmoc-Cys(Trt)-OH was followed by HPLC of the reaction solution. The reaction was interrupted by washing at the stage when 54 mmol was incorporated (˜5 h), corresponding to roughly 0.6 mmol/g. Loading was found to be 0.42 mmol/g.

From this Cys resin, we made the intermediate Fmoc(7-13) resin, which is common to all the (3-13) candidates proposed for ring-closure on resin.

Based on Fmoc loading (0.199 mmol/g), the yield of Fmoc(7-13)-2-CT resin (61.9 g) was 81.6%. The purity of the cleaved Fmoc(7-13)OH, without side chain protection was around 85% (as measured by HPLC).

Some peptide could possibly be cleaved off by the prolonged action of slightly acidic Oxyma as the ester linkage to 2-CT is sensitive to acid. Treatment of Fmoc(7-13)-2-CT resin with about 3 eq of Oxyma in DMF for 21 h (20-25° C.) caused however no loss of peptide.

DMF was used throughout, but NMP was also tested when performing couplings. The latter solvent gave no improvement.

D-H-Cys(Trt)-2-CT (0.40 mmol/g) and [D-H-Cys₁₃(Trt)]Fmoc(7-13)-2-CT (0.175 mmol/g) resins were made in the same way as the L-Cys₁₃ versions.

### Fmoc(3-13)-2-CT Resins

Syringe syntheses of Fmoc(3-13)-2-CT peptides were performed, each from 7.5 g Fmoc(7-13) resin. Couplings were made with Oxyma/DIC for 3-5 h with extra DIC added after approx. 2 h.

| | |
|---|---|
| Fmoc-Glu(OtBu)-Leu-Cys(Trt)-Cys(Trt)-Asn(Trt)- | 8.82 g (98%); 0.158 |
| Val-Ala-BB-Tyr(tBu)-Gly-Cys(Trt)-2CT resin | mmol/g (95.8%) |
| Fmoc-Glu(OtBu)-Leu- [symbol] -Cys(Trt)-Asn(Trt)-Val- | 8.15 g (94%); 0.120 |
| Ala-BB-Tyr(tBu)-Gly-Cys(Trt)-2CT resin | mmol/g (69.6%) |
| Fmoc-Glu(OtBu)-Leu-Cys(Trt)- [symbol] -Asn(Trt)-Val- | 8.11 g (94%); 0.145 |
| Ala-BB-Tyr(tBu)-Gly-Cys(Trt)-2CT resin | mmol/g (83.5%) |
| Fmoc-Glu(OtBu)-Leu- [symbol] -Asn(Trt)-Val-Ala-BB- | 7.79 g (94%); 0.145 |
| Tyr(tBu)-Gly-Cys(Trt)-2CT resin | mmol/g (80.6%) |

[symbol]

indicates data missing or illegible when filed

Yield based on theoretical product weight.

After final washing/drying, small samples were cleaved/deprotected, and subjected to further analyses. Best result was seen using Cys(Trt) in both 5 and 6 positions. Coupling to an already incorporated Thz was slower (4-5 h). The Thz(Me)₂ variant lacks Fmoc-Gly-Leu.

In an attempt to overcome the reluctance of the H-Thz(Me₂)₅-Cys(Trt)-Asn(Trt)-Val-Ala-BB-Tyr(tBu)-Gly-Cys(Trt)-2-CT resin to continued coupling, it was again swelled in DMF and treated with HATU/NMM and Fmoc-Leu-OH at 30° C. overnight. Activated Fmoc-Leu-OAt is formed, and is believed to be more reactive towards hindered amines. Unfortunately, this met with no success.

The estimated yield of the baseline Fmoc-Glu(OtBu)-Leu-Cys(Trt)-Cys(Trt)-Asn(Trt)-Val-Ala-BB-Tyr(tBu)-Gly-Cys(Trt)-2CT resin was 48% (based on Fmoc loadings and purities of (7-13) and (3-13) intermediates, rel. to H-Cys(Trt)-2-CT resin.

### Ring-Closure on Resin (Lactamisation)

Cyclization was made by first removing Fmoc from (3-13) resins, followed by Pd catalysed de-allylation of Hcys, and HATU assisted cyclization (coupling of -Hcy-OH to H-Glu-) for 4-5 h. The reaction steps were done with the following resins:
Fmoc-Glu(OtBu)-Leu-Cys(Trt)-Cys(Trt)-Asn(Trt)-Val-Ala-BB-Tyr(tBu)-Gly-Cys(Trt)-2CT resin (baseline resin)
Fmoc-Glu(OtBu)-Leu-Cys(Trt)-Cys(Trt)-Asn(Trt)-Val-Ala-BB-Tyr(tBu)-Gly-D-Cys(Trt)-2CT resin
Fmoc-Glu(OtBu)-Leu-Thz₅-Cys(Trt)-Asn(Trt)-Val-Ala-BB-Tyr(tBu)-Gly-Cys(Trt)-2CT resin
Fmoc-Glu(OtBu)-Leu-Cys(Trt)-Thz₆-Asn(Trt)-Val-Ala-BB-Tyr(tBu)-Gly-Cys(Trt)-2CT resin
Fmoc-Glu(OtBu)-Leu-Thz₅-Thz₆-Asn(Trt)-Val-Ala-BB-Tyr(tBu)-Gly-Cys(Trt)-2CT resin

Among the all-L analogues, the baseline resin resulted in the purest lactam, although Kaiser test did not signal any profound difference in cyclization rate. Recall that the purities of the linear Thz resins were somewhat lower compared to the baseline resin, due to incomplete couplings.

The estimated yield of the baseline resin was 32% (rel. to H-Cys(Trt)-2-CT resin), as based on Fmoc/Trt loadings and purities of (7-13), (3-13), and (1-13) intermediates.

From the baseline resin (5.41 g), a lactam, shown below, was obtained in amount of 1.80 g upon cyclisation, followed by cleavage/deprotection/precipitation.

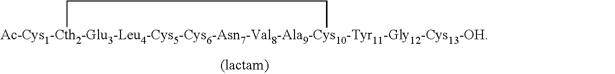

The measured content of D-Cys in the baseline lactam indicates that epimerisation of Cys₁₃ at least is not very pronounced.

### Oxidations (Formation of S—S Bridges)

Oxidation is the final step.

The baseline Ac(1-13)-OH cyclisation product is sufficiently soluble in DMF for HPLC analyses, but it appears difficult to make 10 g/L solutions. It seems very insoluble in MeCN, 20-80% MeCN (aq, w or w/o AcOH), neat AcOH, 25% AcOH(aq), water, EtOH, and MeOH. It is very soluble in DMSO, and the solution is stable when kept at 5-10° C., and even at 25° C. for a few days (HPLC). Ten times dilution in 20% MeCN (aq) hazens the resulting mixture somewhat, but there was no peptide loss upon filtration (HPLC). It is easily dissolved in NMP (10 g/L), but reacts immediately.

The alkaline DMSO oxidation was the most straightforward method. Lowering the crude concentration from 3 to 1 g/L gave practically the same results; no increase in purity was observed compared to the 3 g/L experiment. Irrespective of the concentration of crude lactam, there was no change in HPLC profiles upon prolonging the folding time from 1 to 2 days.

It was not clear whether GSH/GSSG actually had an effect on folding upon iodine oxidation, or if only increased pH changed the HPLC profile; at least in the case of alkaline DMSO oxidation, GSH-GSSG played no discernible role.

NMP as a solvent was not useful, as the peptide reacted very quickly into an undefined mixture. The high reactivity is most likely due to NMP(5-OOH), which is formed in NMP on ageing under oxidative conditions (air).

### CONCLUSIONS

The strategy presented in FIG. 1 can be used to synthesize the synthetic peptide of SEQ ID NO:1, e.g., a standard SPPS protocol to assemble the amino acid into the desired sequence, followed by cyclization through HATU assisted amide bond formation between Hcys and Glu to furnish the macrocyclic, protected peptide on resin shown below
Ac-Cys(Trt)-Hcys(S*)-Glu(OtBu)-Leu-Cys₅(Trt)-Cys₆(Trt)-Asn(Trt)-Val-Ala-Cys*-Tyr(tBu)-Gly-Cys(Trt)-2CT resin
   Hcys2 is linked to Cys10 via a thioether bridge Baseline lactam resin

The estimated yield of this cyclic peptide on resin based on H-Cys(Trt)-2-CT resin was 32%. This corresponds to a cyclisation yield of 67%.

Analogues with Thz in the 5 or 6 positions, or in both, provided no advantage. Couplings onto already incorporated Thz require more time for completion. Substitution with these Cys analogues would furthermore need an extra process step before disulfide bridge construction, to remove the methylene bridge with aqueous MeONH₂ to scavenge formaldehyde. They were not deprotected by TFA cocktails.

Another Cys derivative, Fmoc-Thz(Me)₂-OH, was also explored. It is easily converted into Cys by TFA deprotection, but when built into the peptide sequence, no further couplings were possible.

The cyclized baseline resin shown above is thus the best choice. Without Thz in intermediate peptide sequences, an analytical method to detect possible residual formaldehyde would be unnecessary.

Upon ring-closure, the lactam shown below was obtained by cleavage/deprotection/precipitation (1.80 g from 5.41 g of baseline resin):

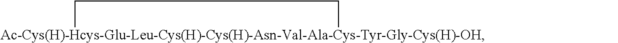

which can also be represented by the following formula:

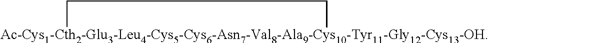

Oxidations, e.g., formation of disulfides from the above lactam, were made either by iodine or in DMSO containing solutions. The correctly folded product was formed (best choice so far was the alkaline DMSO oxidation (HPLC/SEC)).

### OTHER EMBODIMENTS

The present invention is not to be limited in scope by the specific embodiments described herein. Indeed, various modifications of the invention in addition to those described herein will become apparent to those skilled in the art from the foregoing description and the accompanying FIGURE. Such modifications are intended to fall within the scope of the appended claims. It is further to be understood that all values are approximate, and are provided for description.

All patents, patent applications, publications, product descriptions, and protocols are cited throughout this application, the disclosures of which are incorporated herein by reference in their entireties for all purposes.

What is claimed is:

1. A method of producing a synthetic peptide, or a pharmaceutically acceptable salt thereof, the method comprising:
(i) chemically synthesizing a linear peptide having its C-terminal bound to a solid phase support and a protected amine group at its N-terminal using a plurality of amino acids and at least one polyamino acid synthon, the linear peptide having protecting groups in one or more amino acids and/or the at least one polyamino acid synthon;
wherein the synthon has at least one amine group acetylated and at least one carboxylic acid protecting group;
(ii) removing the carboxylic acid protecting group of the synthon and the protecting group from the amine group at the N-terminal of the linear peptide to form a partially unprotected solid phase support-bound peptide having an unprotected amine group and an unprotected carboxylic acid group;
(iii) coupling the unprotected amine group and the unprotected carboxylic acid group to form a cyclized solid phase support-bound peptide;
(iv) cleaving the cyclized solid phase support-bound peptide from the solid phase support to generate a cyclized protected peptide;
(v) globally deprotecting the cyclized protected peptide to obtain a globally deprotected peptide;
(vi) folding the globally deprotected peptide to form one or more additional crosslinks to obtain the synthetic peptide; and
(vii) purifying the synthetic peptide;
wherein the synthetic peptide comprises the amino acid sequence:
Ac-Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1);
wherein the synthetic peptide contains a covalent bond between the following amino acid residues:
a) Cys₁ and Cys₆,
b) Cth₂ and Cys₁₀, and
c) Cys₅ and Cys₁₃.

2. The method of claim 1, further comprising:
lyophilizing the synthetic peptide from solution.

3. The method of claim 1, wherein the solid phase support is selected from the group consisting of Wang resins, Trityl resins, and Rink resins.

4. The method of claim 1, wherein the solid phase support has a loading of about 0.10 mmol/g, about 0.20 mmol/g, about 0.30 mmol/g, about 0.40 mmol/g, about 0.50 mmol/g, about 0.60 mmol/g, about 0.70 mmol/g, about 0.80 mmol/g, about 0.90 mmol/g, or about 1.00 mmol/g.

5. The method of claim 5, wherein the polyamino acid synthon is a compound of the formula: 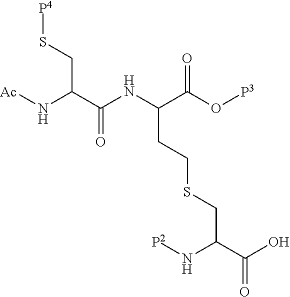
wherein:
P² is an amine protecting group;
P³ is a carboxylic acid protecting group; and
P⁴ is a thiol protecting group.

6. The method of any one of claims 1-5, wherein the protecting groups are selected from the group consisting of fluorenylmethyloxycarbonyl (Fmoc), tert-butyloxycarbonyl (Boc), carboxybenzyl (Cbz), trityl, methyl, ethyl, tert-Butyl, allyl, 2,4-dimethoxybenzyl (Dmb), 9-fluorenylmethyl (Fm), benzyl (Bn), tert-butyldimethylsilyl, allyloxycarbonyl (alloc), tert-butyloxycarbonyl, acetamidomethyl (Acm), 3-nitro-2-pyridine sulfenyl (NPYS), and 2-pyridine-sulfenyl (Pyr).

7. The method of claim 5 or 6, wherein P² is a 9-fluorenylmethoxycarbonyl (Fmoc) protecting group.

8. The method of any one of claims 5-7, wherein P³ is an allyl protecting group.

9. The method of any one of claims 5-8, wherein P⁴ is a trityl protecting group.

10. The method of any one of claims 1-9, wherein the subunits of the polyamino acid synthon have a D-configuration.

11. The method of any one of claims 1-9, wherein the subunits of the polyamino acid synthon have an L-configuration.

12. The method of any one of claims 1-11, wherein the subunits of the polyamino acid synthon have both a D-configuration and an L-configuration.

13. The method of any one of claims 1-12, wherein the plurality of amino acids and the synthon are coupled by a carbodiimide-mediated reaction or by a reaction mediated by a non-carbodiimide coupling agents: 1-[Bis(dimethylamino)methylene]-1H-1,2,3-triazolo[4,5-b]pyridinium 3-oxide hexafluorophosphate (HATU), (2-(1H-benzotriazol-1-yl)-1,1,3,3-tetramethyluronium hexafluorophosphate (HBTU), 1H-Benzotriazolium 1-[bis(dimethyl-amino)methylene]-5-chloro-hexafluorophosphate (1-),3-oxide (HCTU), O-(Benzotriazol-1-yl)-N,N,N′,N′-tetramethyluronium tetrafluoroborate (TBTU), benzotriazol-1-yloxytripyrrolidinophosphonium hexafluorophosphate (PyBOP), 7-Azabenzotriazol-1-yloxy)tripyrrolidinophosphonium hexafluorophosphate (PyAOP), or propanephosphonic acid anhydride (T3P) to form the linear peptide of step (i).

14. The method of any one of claims 1-13, wherein at least one amino acid from the plurality of peptides and/or the synthon are coupled by a carbodiimide-mediated reaction to form the linear peptide of step (i).

15. The method of claim 14 or 15, wherein the carbodiimide is selected from the group consisting of diisopropylcarbodiimide (DIC), dicyclohexylcarbodiimide (DCC) and 1-Ethyl-3-(3-dimethylaminopropyl)carbodiimide (EDC).

16. The method of 15, wherein the carbodiimide is DIC.

17. The method of any one of claims 15-18, wherein the carbodiimide-mediated reaction further comprises an antioxidant.

18. The method of claim 17, wherein the antioxidant is 1,3-diisopropyl-2 thiourea (DITU) or dithiothreitol.

19. The method of any one of the preceding claims, wherein the cyclization coupling reaction is mediated by a non-carbodiimide coupling agent.

20. The method of claim 19, wherein the non-carbodiimide coupling agent is HATU.

21. The method of any one of claims 1-20, wherein the cyclized peptide of step (iii) contains a thioether bond.

22. The method of any one of claims 1-21, wherein the global deprotection step (v) comprises addition of a cocktail solution having ammonium iodide (NH₄I).

23. The method any one of claims 1-22, wherein the folding step (vi) is achieved via an iodine- or alkaline-mediated oxidation.

24. The method of claim 23, wherein the alkaline-mediated oxidation is a dimethylsulfoxide (DMSO)- or an N-Methyl-2-pyrrolidone (NMP)-mediated oxidation.

25. The method of any one of claims 1-24, wherein the covalent bond between Cys₁ and Cys₆ and Cys₅ and Cys₁₃ is a disulfide bond.

26. A method of preparing a synthetic peptide of Formula I: 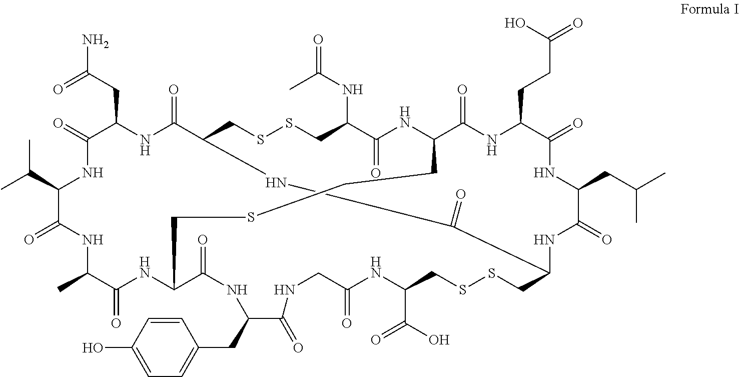 [SMILES: CC(=O)N[C@@H]1CSSC[C@@H]2NC(=O)[C@@H]3CSSC[C@@H](C(=O)O)NC(=O)CNC(=O)[C@@H](Cc4ccc(O)cc4)NC(=O)[C@@H](CSCC[C@@H](NC1=O)C(=O)N[C@@H](CCC(=O)O)C(=O)N[C@@H](CC(C)C)C(=O)N3)NC(=O)[C@@H](C)NC(=O)[C@@H](C(C)C)NC(=O)[C@@H](CC(N)=O)NC2=O]
comprising:
(i) coupling a C-terminal resin bound Tyr-Gly-Cys peptide having protected amino acid side chains to a polyamino acid synthon of Formula II: 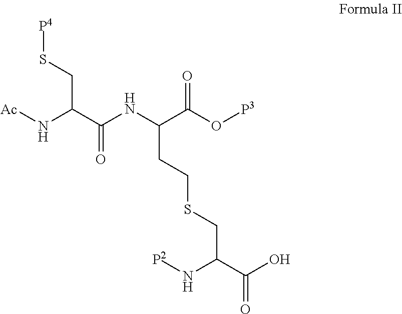
wherein:
P² is an amine protecting group;
P³ is a carboxylic acid protecting group; and
P⁴ is a thiol protecting group;
to form a resin bound peptide of Formula III: 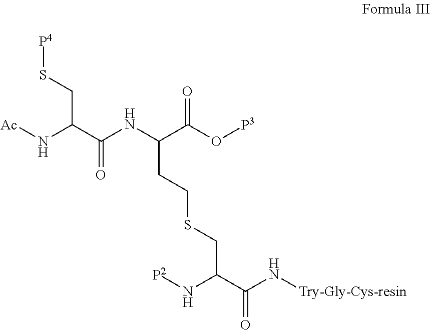
(ii) removing the P² protecting group of Formula III to obtain a resin bound peptide of Formula IV having an unprotected amine group: 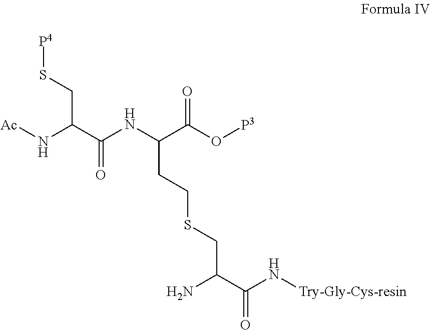
(iii) coupling a P²-alanine to the resin bound peptide of Formula IV via the free amine group of Formula IV to form a resin bound peptide of Formula V: 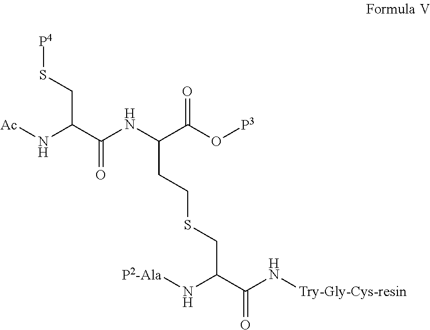
(iv) removing the P² protecting group of Formula V to obtain a free amine group, followed by coupling the free amine group to P²-amino acid,
wherein the side chain of the P² amino acid may be protected;
(v) repeating step (iv) for five more times to form a resin bound peptide of Formula VI: 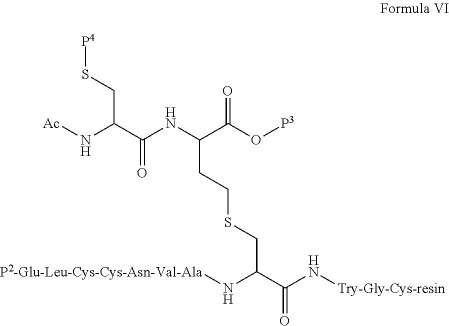
wherein at least one amino acid side chain is protected;
(vi) removing the P² protecting group and the P³ protecting group to obtain a free amine group and a free carboxylic acid group;
(vii) coupling the free amine group and the free carboxylic acid group, to obtain a cyclized peptide of Formula VII: 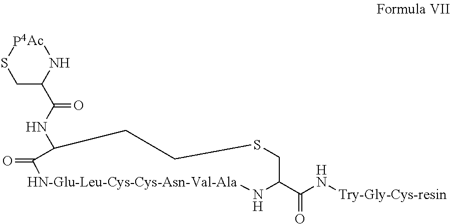
(viii) cleaving the peptide of Formula VII from the resin to obtain a cyclized peptide;
(ix) globally deprotecting the cyclized peptide to obtain a globally deprotected peptide; and
(x) folding the globally deprotected peptide by forming two disulfide bonds to obtain the synthetic peptide of Formula I.

21. The method of claim 20, wherein the Glu, Cys, Cys, Asn, Gly, and Cys residues of Formula VI have side chain protecting groups.

22. The method of claim 20 or 21, wherein P² is a protecting group selected from the group consisting of fluorenylmethoxycarbonyl (Fmoc), tert-butyloxycarbonyl (Boc), carboxybenzyl (Cbz), and allyloxycarbonyl (Alloc).

23. The method of claim 22, wherein P² is a fluorenylmethoxycarbonyl (Fmoc) protecting group.

24. The method of any one of claims 20-23, wherein P³ is a protecting group selected from the group consisting of methyl, ethyl, tert-Butyl, allyl, trityl, 2,4-dimethoxybenzyl (Dmb), 9-fluorenylmethyl (Fm), and benzyl (Bn).

25. The method of claim 24, wherein P³ is an allyl protecting group.

26. The method of any one of claims 20-25, wherein P⁴ is a protecting group selected from the group consisting of acetamidomethyl (Acm), tert-butyl (t-But), 3-nitro-2-pyridine sulfenyl (NPYS), 2-pyridine-sulfenyl (Pyr), and trityl (Trt).

27. The method of claim 26, wherein P⁴ is a trityl protecting group.
